(19) United States
(12) Patent Application Publication
(10) Pub. No.: US 2004/0006396 A1
(43) Pub. Date: Jan. 8, 2004

(54) Transcutaneous devices having nueral interface

(76) Inventors: John L. Ricci, Middletown, NJ (US); Harold Alexander, Short Hills, NJ (US)

Correspondence Address: Melvin K Silverman, Suite 500, 500 West Cypress Creek Road, Fort Lauderdale, FL, 33309, US

(21) Appl. No.: 10/237,355

(22) Filed: Sep. 6, 2002

Related U.S. Application Data
(63) Continuation-in-part of application No. 09/784,284, filed on Feb. 16, 2001.
(63) Continuation-in-part of application No. 09/784,296, filed on Feb. 14, 2001.
(63) Continuation-in-part of application No. 09/500,038, filed on Feb. 8, 2000, now Pat. No. 6,419,491.
(63) Continuation-in-part of application No. 08/996,224, filed on Dec. 22, 1997, now Pat. No. 6,147,666.
(63) Continuation-in-part of application No. 08/639,712, filed on Apr. 29, 1996, now abandoned.
(63) Continuation of application No. 08/390,805, filed on Feb. 15, 1995, now abandoned.
(63) Continuation of application No. 08/146,790, filed on Nov. 2, 1993, now abandoned.
(60) Provisional application No. 60/317,568, filed on Sep. 6, 2001.

(51) Int. Cl. A61F 2/28; A61F 2/78; A61B 17/86

(52) U.S. Cl. 623/32; 623/23.5; 606/72; 604/175; 623/25

(57) ABSTRACT

A transcutaneous implant constitutes proximal partially external section; a collar section, integrally dependent from the external section, the collar section having a microtextured surface constituting a microgeometric, repetitive surface pattern, in a form of multiplicity of alternating ridges and grooves, each having an established width in a range of about 2 to about 25 microns, and an established depth in a like range; an anchor-like distal section, integrally dependent from the collar section, for implantation into a bone segment to be stabilized during a medically advantageous period of time. The surface pattern defines a guide at tissue, cellular and molecular levels for a preferential promotion of rate, orientation and direction of growth of colonies of cells which are in contact with the pattern.

### REFERENCE TO RELATED APPLICATIONS

This Application is a utility conversion of Provisional Patent Application No. 60/317,568, filed Sep. 6, 2001, under 35 U.S.C. 119(e), and the same is incorporated in its entirety by reference. This application is also a continuation-in-part of application Ser. No. 09/784,296, filed Feb. 16, 2001 which in turn is a continuation-in-part of application Ser. No. 09/500,038, filed Feb. 8, 2000 which is a continuation-in-part of application Ser. No. 08/996,224, filed on Dec. 22, 1997, now abandoned, which is a continuation-in-part of application Ser. No. 08/639,712, filed on Apr. 29, 1996, now abandoned, which is a continuation of application Ser. No. 08/390,805, filed on Feb. 15, 1995, now abandoned, which is a continuation of application No. 08/146,790, filed on Nov. 2, 1993, now abandoned.

N/A

### BACKGROUND OF THE INVENTION

Transcutaneous devices have application in many areas of medicine, these including such areas as bone stents or pins where it is necessary to stabilize fragments of a bone during a period of healing, medication-dispensing soft tissue implants such as those that require external access by the needle of a syringe, and neural interface devices that require both physical stability at an amputation and/or nerve injury site and an external electrical port to which a prosthesis is may be attached. These unique and diverse requirements, of transcutaneous devices have presented long-standing challenges within the medical disciplines, in which they exist.

Most transceutenous devices must satisfactorily address requirements at three levels, namely, bone interface stability, soft tissue stability, and suitable properties of an external portion thereof. Accordingly, in a successful transcutaneous device, it is necessary to obtain a stable, reliable and bacteria free interface to both the hard and soft tissue surfaces thereof.

The instant invention employs a selectable surface micro-texturing of respective interfaces of the implant to achieve ingrowth of skin, connective tissue, and bone to its surfaces. As such, this invention addresses a long-felt need within areas of medicine that employ such implants.

### SUMMARY OF THE INVENTION

The inventive subcutaneous implant, when used in simple orthopedic applications such as assuring stability of segments of a broken bone, employs a lateral, circumferential region of micro-texturing upon the stent or bone pin which is in contact with the skin. An implant, in accordance with this embodiment, includes an external or proximal section, a collar section upon which the micro-texturing is applied, and a distal or anchor-like section which is implanted within the bone segments to be stabilized during a period of healing. Such texturing of the collar section constitutes a micro geometric, repetitive surface pattern in a form of a multiplicity of alternating ridges and grooves, each having an established width in a range. of about two to about twenty-five microns, and an established depth in a like range. Such surface patterns define a guide at tissue, cellular, and molecular levels, for a preferential promotion of the rate, orientation and direction of growth of colonies of cells which are in contact with said pattern. In the orthopedic application, the distal or anchor-like section of the implant is furnished with a smooth surface to enable the physician to easily remove the stent or pin from the bone after healing of the fracture has occurred.

In an implantable device for the dispensing of micro-quantities of medication, the above pattern of texturing is employed to assure stability of the pharmacologic-agent containing implant within soft tissue while assuring that an external portion thereof is not subject to uncontrollable cell growth which would otherwise result thereby covering the syringe needle input to the dispensing device.

In a neural interface embodiment of the invention, a “neural port,” having the external appearance of the anchor of a dental implant, has an anchor portion thereof embedded within bone at an amputation site while a collar portion thereof is provided with a transverse channel in which is embedded muscle tissue and surviving nerves associated therewith. This combination of muscle and nerve tissue is embedded in a hollow conductive cylinder which itself is transversely embedded within said channel of said collar portion of the neural interface, thereby achieving electrical communication of the nerves within said muscles, to said cylinder and to the collar of the interface. Stability of said collar within surrounding soft tissue is achieved through a pattern of surface micro-texturing. Electrical communication by the prosthesis with the neural interface is achieved through an axial port in the interface into which is placed a prosthesis-associated electrode to thereby achieve electrical communication with said nerves of said muscle embedded within said conductive cylinder.

It is accordingly an object of the invention to provide a transcutaneous implant having improved means of anchoring the same to soft and/or hard tissue of a patient within which it is embedded.

It is another object to provide a transutaneous implant having particular application as a bone pin or stent for use in the stabilization of fractures during the healing period thereof.

It is a further object of the invention to provide an improved means of anchoring small transcutenous drug delivery means within soft tissue.

It is a still further object to provide a neural prosthetic interface having enhanced soft and hard tissue stability with resultant improved electrical communication between and within such interface and nerve sites associated with a trauma or amputation.

The above and yet other objects and advantages of the present invention will become apparent from the hereinafter set forth Brief Description of the Drawings and Detailed Description of the Invention.

### BRIEF DESCRIPTION OF THE DRAWINGS

FIG. 1 is a perspective view of an orthopedic implant having particular application as a bone stent of pin.

FIG. 2 is a radial cross-sectional through Line 2-2 of FIG. 1.

FIG. 3 is a view of the implant of FIG. 1 showing the same implanted in a fractured bone to secure together bone elements of the fracture.

FIG. 4 is a schematic view showing a device for long-term subdermal delivery of micro-quantities of a pharmacologic agent.

FIG. 5 is a perspective view thereof showing use of the inventive micro texturing upon the surface thereof.

FIG. 6 is a perspective view of a first embodiment of a neural prosthetic interface in accordance with the present invention.

FIG. 7 is an in vivo view of the interface of FIG. 6 and an associated annular element employed during surgery to stabilize the interface within soft tissue.

FIG. 8 is an enlarged partial breakaway of the upper portion of FIG. 7.

FIG. 9 is a scanning electron micrograph showing the appearance of 12 mm wide grooves machined into a surface of implants of any of the embodiments of the invention.

FIG. 10 is a photograph showing the external appearance of an electrical port resultant from the neural prosthetic interface.

FIG. 11 is a cross-sectional view of a further embodiment of the neural prosthetic interface shown in association with a healing cap employed therewith.

FIGS. 12 thru 17 are schematic views showing xy surface patterns of alternating ridges and grooving that may be employed for microtexturing within the scope of the present invention.

FIGS. 18 thru 25 are vertical or radial cross-sectional yz plane views showing the depth-associated geometry of ordered microgeometric repetitive surfaces of the types shown in FIGS. 12 thru 17.

### DETAILED DESCRIPTION OF THE INVENTION

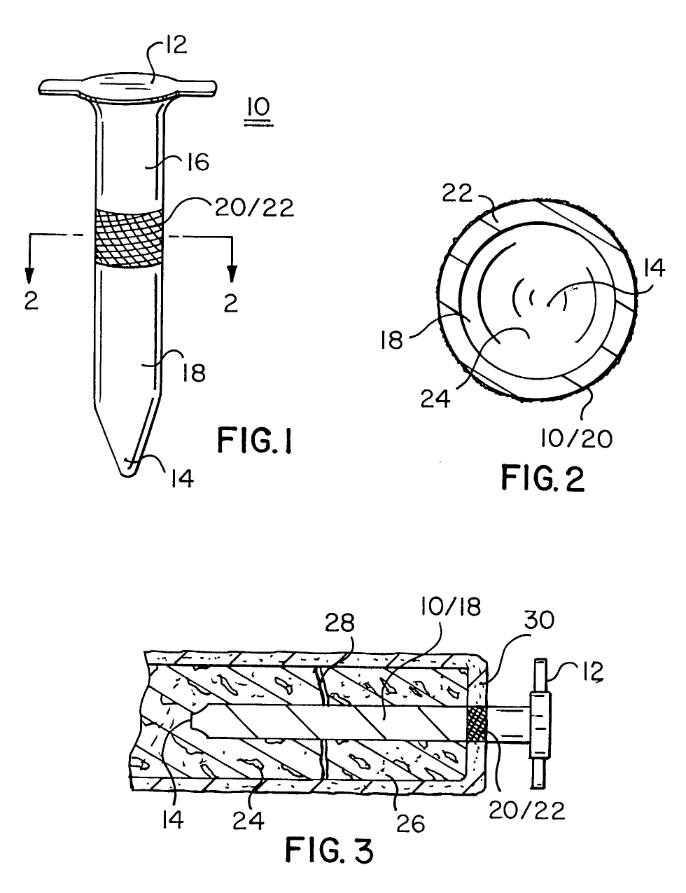

With respect to the perspective view of FIG. 1, there is shown a bone stent or pin 10 which comprises a free or handle 12, an insertion tip 14, a neck 16, a hard tissue interface portion 18, and a collar 20. Said pin is shown in radial cross-sectional view in FIG. 2. It may be appreciated that the stent is typically the handle 12 and is visible only externally after its insertion

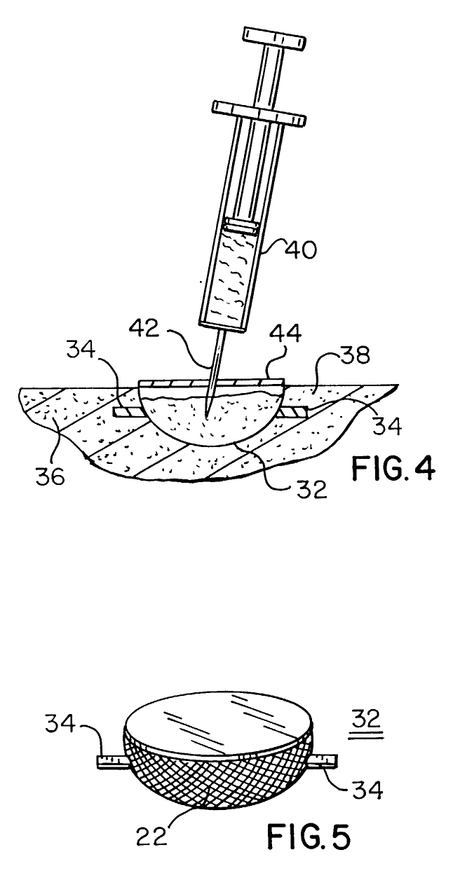

The characterizing aspect of the bone pin may be appreciated with reference to FIG. 3, which shows a microgeometric repetitive surface pattern 22 which has been applied to collar 20. Said collar is positioned so that bone pin 10 when properly positioned between elements 24 and 26 of a bone fracture 28 will hold the elements thereof together during a healing period. Therein most of the length of pin 18, for example, tip 14 and hard tissue interface portion 18 are entirely smooth such that no ingrowth of bone from segments 24 or 26 can grow into a polished surface thereof. It is noted that implants of this type are typically formed of an alloy of titanium. However, about collar 20 thereof is formed said micro-textured surface pattern 22 which, it has been determined through experimentation will induce ingrowth of soft tissue 30 of the dermis and epidermis of the patient, thereby stabilizing pin 10 during the period of healing. Thereafter, the treating physician is easily able to effects small cuts about the micro textured area and then rotate handle 12 to remove pin 10 from the then healed bone. It has been found that such microtexturing is preferably in the nature of a repetitive surface pattern in a form of multiplicity of alternating ridges and groove, each having an established width in a range of about 2 to about 25 microns, and an established depth within a similar range. In the case of soft tissue ingrowth, ridges and grooves having widths and depths in a range of A to B have been found to be preferably. Such a microgeomtric repetitive pattern defines a guide for the preferential promotion of the rate, orientation and direction of growth of colonies of cell of the tissue 30 which are in contact with said pattern.

With reference to the views of FIGS. 4 and 5, another application of the invention relative to a transcutaneous device 32 for the delivery of micro or nano-quantities of drugs 33 is shown. It should be noted, that the device 32 is implanted within soft tissue 36 and mechanically stabilized by an annular ring 38. Replenishment of the contents of the dispensing device, which often is in the nature of a micro-mechanical or osmotic pump (not shown), occurs through the use of a syringe 40, a needle 42 of which is used to penetrate an elastomeric cover 44 at a point of entry that is essentially imperceptible after the needle is removed.

In devices of the above type, a problem is that of stability within tissue 36. Accordingly, the above described micro-texturing 22 may be applied to the outer surface thereof as is shown in FIG. 5 to assure maximum ingrowth of tissue 36 and, further, define the rate, orientation and directionality of such growth as is deemed clinically optimal by the prescribing physician.

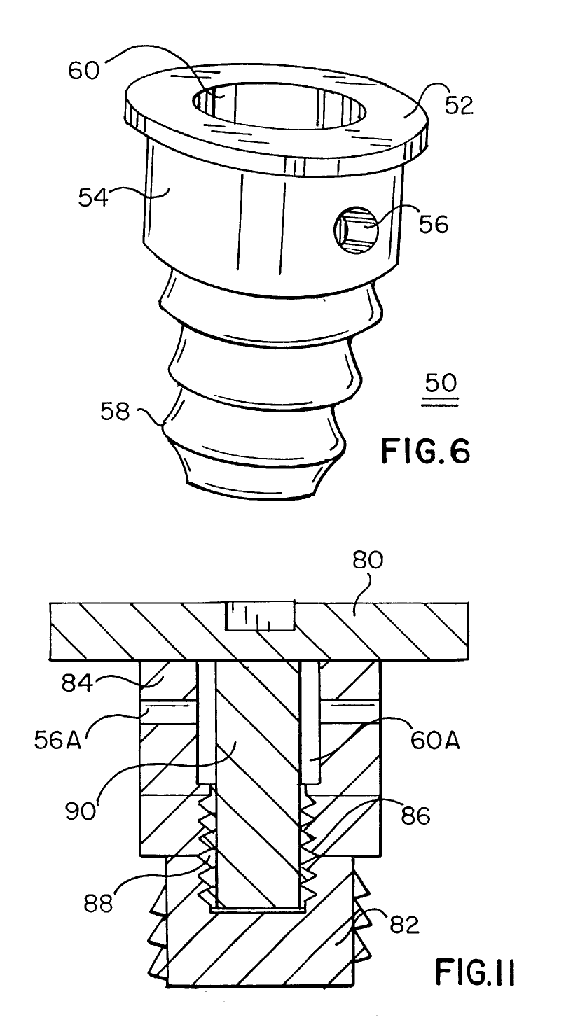

With reference to FIGS. 6 thru 11, the present invention is shown with reference to a neural prosthetic interface 50 which is characterized by a free or proximal end 52, a collar 54, a transverse channel 56 within said collar, and an anchor 58. The interface 50 further includes an axial channel 60 which communicates downwardly into at least as far as transverse channel 56. Interface 50 is intended to define an integrated neuromuscular platform and transcuteneous port which, as noted in the Background of the Invention above, may serve as a basis of electrical communication between functional elements of a prosthesis and an amputation or trauma site, this as is more fully set forth below.

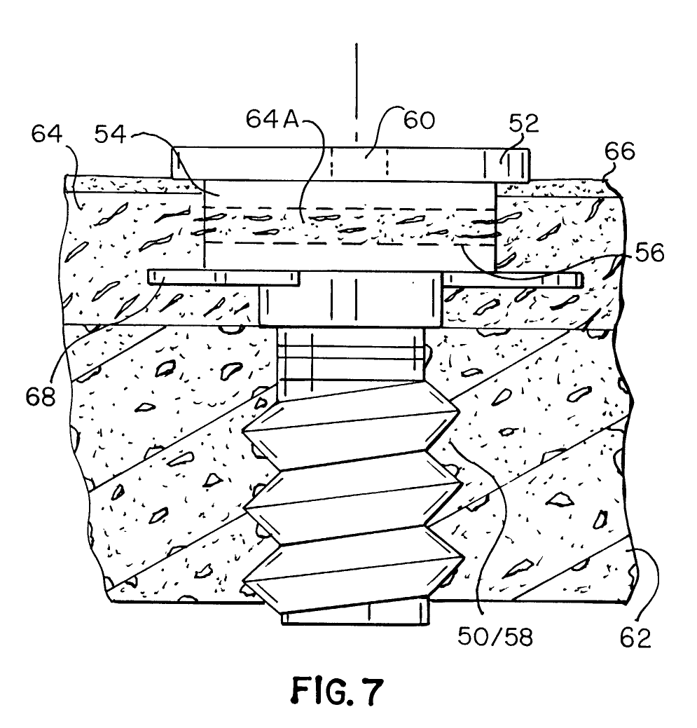

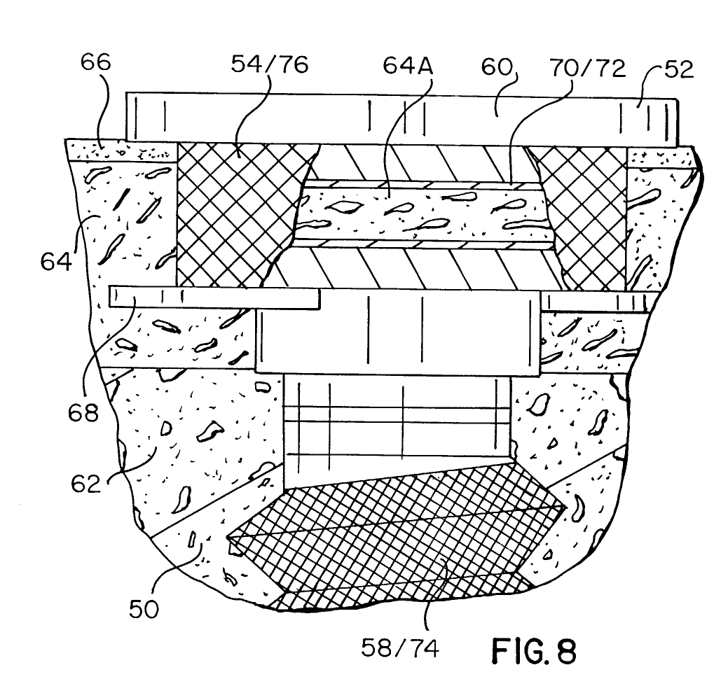

The neural interface is shown in vivo in the cross-sectional view of FIG. 7. FIG. 8 is an enlargement and partial breakaway of the collar portion of FIG. 7 Therein, interface 50 may be seen implanted within bone 62, subcutaneous tissue 64, and epithelial tissue 66. Anchor 58 stabilizes the interface within bone 62 while collar portion 54 and a surgical washer 68 secure and stabilize the interface within the subcutaneous and epithelial tissue. This subcutaneous tissue is typically in the nature of muscle which is inherently rich in nerve density and, as such, constitutes an ideal candidate for use in a neural interface. Within channel 56 is inserted a neural guide 70 (see FIG. 8) which is in the nature of a silicone or silastic tube containing therein spiral lead wires 72 or conductive doping. Nerve guide 70 thereby encapsulates nerve dense muscle tissue 64a and provides means of electrical communication therewith. Inasmuch as collar 54 is also formed of a conductive material, electrical communication between nerves muscle tissue 64a and collar 64 of the interface 50 may be achieved.

It is to be appreciated that other nerve guides or nerve guide tubes may be employed within channel 56 of collar 54. For example, U.S. Pat. No. 5,656,605 (1977) to Hansson, et al, entitled Device to Provide Drug-induced Regeneration, teaches a nerve guide tube containing a therapeutic composition having a nerve growth-stimulating agent disbursed within a matrix forming gel, which nourishes the nerves within tissue 64a. As such, the present invention may be employed with nerve guides of any type having a geometry compatible with said transverse channel 56.

With further reference to FIG. 8, there may be seen a first microtextured pattern 74 upon anchor 58 and a second microtexturing pattern 76 upon collar 54 of the interface. As with the above described other embodiments of the invention, such microgrooving consists of microgeometric, repetitive surface patterns, in the form of alternating ridges and grooves, each having an established width in a range of about two to about twenty five microns, and having an established depth within a like range. It has however been determined that better ingrowth and stability of such surfacing to soft tissue, such as subcutaneous tissue 64, is achieved with a micron pattern toward the lower end of said range, while more effective ingrowth and stability relative to bone 62 is achieved with dimensions closer to the higher end of said range. It has as such been established that the use of such surface micro-texturing is of considerable value in assuring stability of an implant relative to both hard and soft tissue. As may be appreciated, in the present application of a neural interface, it is imperative to assure long term stability in tissue of both types, this due to the functions, both mechanical and electrical, of such an interface.

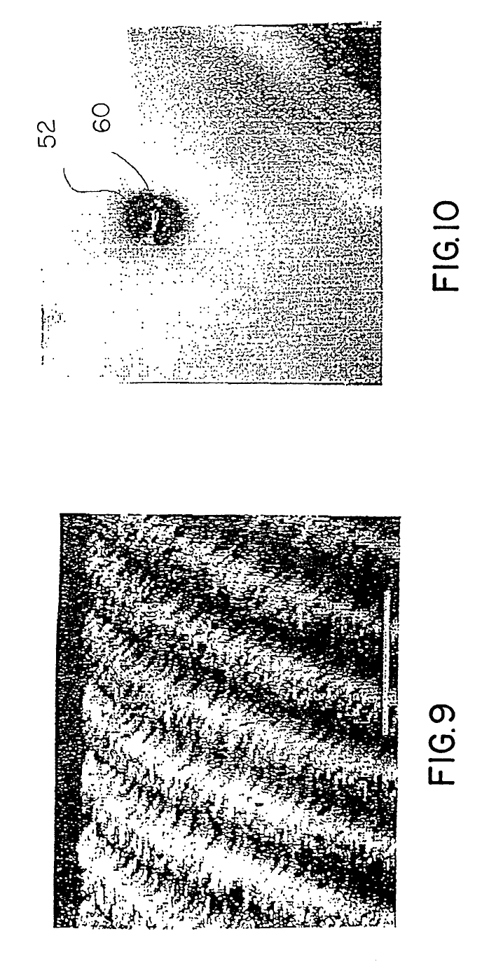

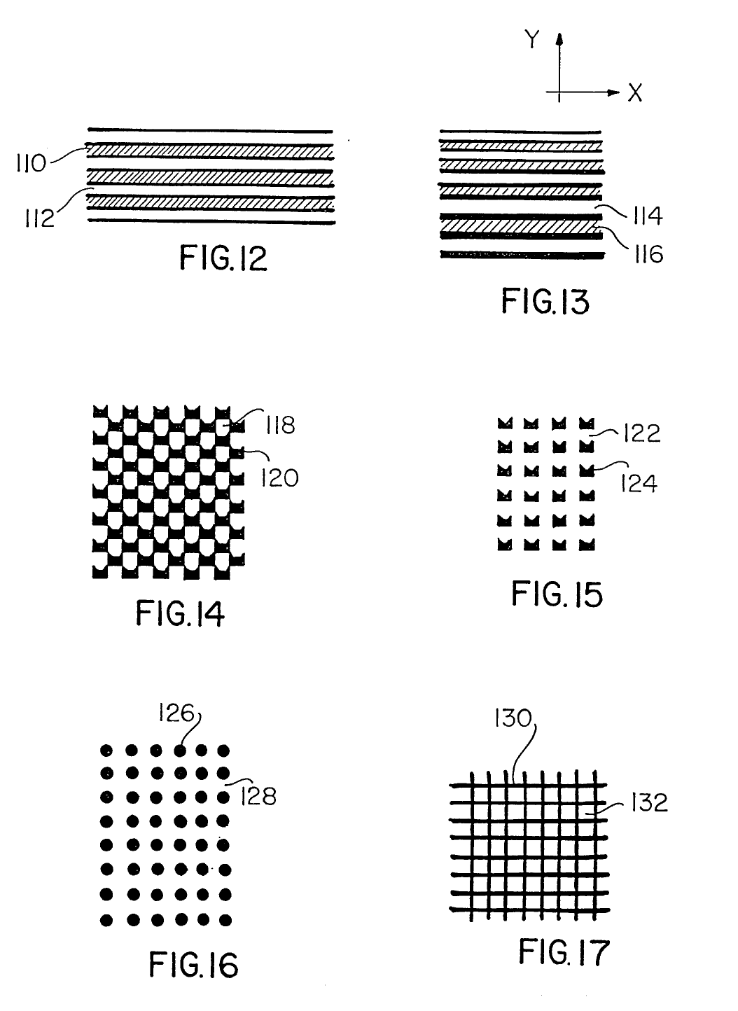

After in vivo implantation of the neural interface has been accomplished in the manner shown in FIGS. 7 and 8, an electrode having electrical communication with functional elements of a prosthesis may be press fittable inserted into axial channel 60 and into electrical communication about nerve guide tube 70, this without regard to which type of nerve guide technology is employed. There is thereby provided a mechanism capable of effecting transfer of low voltage, e.g. 50 microvolt level information, through the skin safely and for an extended period of time. An external view of port 60 and surrounding area 52 of the interface, after in vivo implantation is shown in FIG. 10. The present invention thereby addresses one of a longstanding problems in the present area, that is, the inability of soft tissue to form effective interfaces with typically titanium metal surfaces that have been historically utilized in prosthetic fixation. In combination, therewith, the instant invention provides reliable bone fixation by virtue of the above described surface micro-texturing and its resultant integration into human tissue at levels both cellular and molecular. Micro-texturing in accordance with the present invention is shown in the scanning electron micrograph of FIG. 9 in which each groove has a width of 12 microns. Such micro-machining may be accomplished by a variety of technologies, this including, without limitation, laser micro-machining as is taught by U.S. Pat. No. 5,645,740 to Naiman, et al.

In FIG. 11 is shown an alternate embodiment 78 of the interface of FIGS. 6 thru 8 in which a healing cap 80 is secured within anchor and collar portions 82 and 84 respectively of the neural interface. The cap assures complete and hygienic healing of the external area shown in FIG. 10 at which the port of the interface is to be implanted. Therein, there is provided an axial channel 60a provided, at a distal end thereof, with threads 86 which mate with threads 88 which depend from axial member 90 of the healing cap 80 to thereby assure stability of the cap relative to the implant. In the embodiment of FIG. 11, different types of microtexturing may, as in the embodiments of FIGS. 6 thru 8, be applied to respective hard and soft tissue interfaces.

Further shown in FIG. 11 is transverse channel 56a in which the above described neural guide tube is inserted to provide ultimate electrical communication with an electrode of a prosthesis, to and from which electrical signals are ultimately to be exchanged. Thereby, electrodes originating from the prosthesis will achieve sufficient electrical contact with the interface 78 due to sufficiently small mechanical movement and therewith minimal random noise such that specific signals to and from the interface may be utilized with the prosthesis, even at voltages of about 50 microvolts.

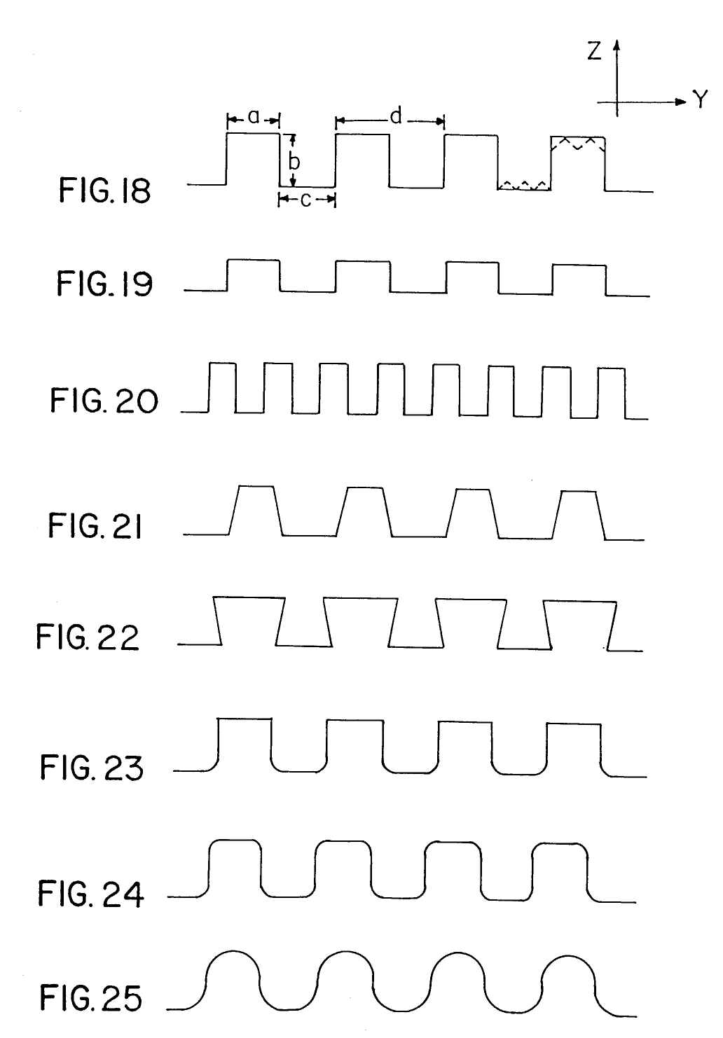

The range of xy plane or surface geometries which may be employed to produce advantageous microtexturing is shown schematically in FIGS. 12 thru 17. Therefrom, it may be appreciated that the referenced grooves 112/114 and ridges 110/114 need not be linear but, as well, may constitute alternating points or “lands” of heights 120, 124, 126, 132 and depressions 118, 122, 128, 130. This is more fully shown with reference to the vertical or radial cross-sectional yz plane views of FIGS. 18 thru 25 which indicate the large variety of geometries which may exist in dimensions a, b, c and d of vertical or radial cross-section through a micro-texturized surface. The selected micro pattern, whether with reference to the surface (xy) plane of FIGS. 12 thru 17 or the vertical/radial (yz) plane of FIGS. 18 thru 25 will of course be dictated by particular cellular characteristics of the tissue of interest. Therein, it has been discovered that a properly selected pattern of repetitive microgrooving will define a guide for a preferential promotion of rate, orientation and direction of growth of colonies of cells of both soft and hard tissue.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

Having thus described our invention, what we claims as new, useful and non-obvious and, accordingly, secure by Letters Patent of the United States is:

1. A transcutaneous implant, comprising:
(a) a proximal partially external section;
(b) a collar section, integrally dependent from said external section, said collar section having a microtextured surface comprising a microgeometric, repetitive surface pattern, in a form of multiplicity of alternating ridges and grooves, each having an established width in a range of about 2 to about 25 microns, and an established depth in a like range;
(c) an anchor-like distal section, integrally dependent from said collar section, for implantation into a bone segment to be stabilized during a medically advantageous period of time,
whereby said surface pattern defines a guide at tissue, cellular and molecular levels for a preferential promotion of rate, orientation and direction of growth of colonies of cells which are in contact with said pattern.

2. The implant as recited in claim 1, in which said distal section comprises a smooth surface to facilitate removal thereof from said bone segment after a healing period thereof.

3. The implant as recited in claim 1, in which:
said bone segment, at which said distal section is implanted, comprises as amputation site; and
said collar section includes a transverse channel therein, in which is embedded muscle and nerve tissue associated with said amputation site.

4. The implant as recited in claim 3, comprising:
a hollow conductive cylinder in which said muscle and nerve tissue are embedded, in which said cylinder is then itself conductively secured within said transverse channel of said collar section,
whereby a neural interface, to which a prosthesis may be joined through said proximal external section, is thereby defined.

5. A transcutaneous implant, comprising:
(a) a proximal partially external section;
(b) a collar section integrally dependent from said external section, implantable within soft tissue; said collar section having a microtextured surface comprising a microgeometric, repetitive surface pattern, in a form of multiplicity of alternating ridges and grooves, each having an established width in a range of about 2 to about 25 microns, and an established depth in a like range;
Said collar section comprising a microdispenser of medication or osmotic pump; and
(c) anchor-like, distal section, integrally dependent from said collar section, for implantation into associated soft tissue to thereby stabilize said collar section therein during a medically advantageous period of time,
whereby said surface pattern defines a guide at tissue, cellular and molecular levels for a preferential promotion of rate, orientation and direction of growth of colonies of cells which are in contact with said pattern.
